(19) United States
(12) Patent Application Publication
(10) Pub. No.: US 2002/0156419 A1
(43) Pub. Date: Oct. 24, 2002

(54) DISPOSABLE MILK COLLECTING BAG FOR A BREAST PUMP

(76) Inventors: BRIAN H. SILVER, CARY, IL (US); GOTTHILF WENIGER, CARY, IL (US)

Correspondence Address: BRINKS HOFER GILSON & LIONE, P.O. BOX 10395, CHICAGO, IL, 60610, US

(21) Appl. No.: 08/203,672

(22) Filed: Feb. 28, 1994

Related U.S. Application Data
(63) Continuation of application No. 07/809,921, filed on Dec. 6, 1991, now abandoned.

(51) Int. Cl. A61M 1/06

(52) U.S. Cl. 604/74

(57) ABSTRACT

A disposable bag for attachment to a breastpump for containing breastmilk has a liquid containing portion with a releasably sealed opening, a tie for re-sealing the opening after use, a writing area formed integral with the bag which does not overlie the liquid containing portion, and an integral pour spout. The liquid containing portion of the bag can be hermetically sealed. The releasable seal provided defines the opening in the bag for attachment of the bag to the breastmilk pump. A tie is fit through two holes in the bag at substantially opposite sides of the opening of the bag so that the opening can be re-sealed when the bag is filled and removed from the breastmilk pump. The integrally formed writing area that is isolated from the liquid containing portion of the bag can be advantageously formed to further define the pour spout that is also integral with the bag. A notch is provided at the periphery of the bag at the pour spout to facilitate tearing of the bag to pour milk from the bag.

### FIELD OF THE INVENTION

The present invention generally relates to breastmilk pumps, and more particularly relates to an improved bag adapted for attachment to a breastmilk pump.

### BACKGROUND OF THE INVENTION

Breastmilk pumps are well known and are generally comprised of a hood that fits over the breast, a vacuum pump connected to the hood for generating an intermittent vacuum within the hood, and a receptacle for the expressed milk. There are manually driven vacuum pumps (e.g., handheld piston pumps) which most commonly connect to at or closely adjacent to the hood, as well as vacuum pumps that are driven by an electric motor and interconnect to the hood via tubing. The vacuum pumps of these devices intermittently generate a vacuum (or a negative pressure) within the hood, with the hood encompassing the nipple and a substantial amount of the breast. The intermittent suction action of the pump serves to pull on the breast, drawing it within the narrowing funnel of the hood, to thereby extract milk in an action reminiscent of suckling. The milk so extracted typically flows from the hood into a container, e.g., a bottle, for storage and later use. A breastpump of the foregoing type is shown in U.S. Pat. No. 4,857,051.

While rigid milk containers (bottles) are most often used with breastpumps, it is also desirable to use disposable plastic bags as the containers.

### SUMMARY OF THE INVENTION

One of the principal objects of the present invention is to provide a sanitary disposable bag for attachment to a breastmilk pump for containing breastmilk that can be easily and efficiently manufactured, packaged and used. To these and other ends, the inventive breastmilk bag comprises an improved flexible plastic bag adapted to contain milk, such as a bag formed by two sheets of plastic constituting a front and a back sheet that are in facial engagement and are joined to each other by a series of seals in such manner to define a hermetically sealable liquid containing portion of the bag. One feature of the invention is a writing area formed integral with the bag by the same sealing technique, but with the existing area isolated from the liquid containing portion of the bag. The bag can accordingly be written on more easily than bags with milk beneath the writing area and without risk of puncturing the milk containing portion.

Another feature of the invention resides in the liquid containing portion of the bag having an opening for attachment of the bag to the breastmilk pump, which opening is releasably sealed, as by a peelable coating, weak heat seal or other appropriate releasable fastening means such that the sealed bag can be readily peeled open for attachment to the breastmilk pump.

The inventive bag also has a tie that can be laced through at least two holes provided at substantially opposite sides of the bag opening. The laced tie is twisted upon itself to re-seal the opening when the bag is removed from the breastmilk pump, as for storage.

Yet another aspect of the invention is a pour spout formed integral with the bag and separate from the foregoing bag opening. The pour spout can be opened to pour contained milk from the bag. In a preferred embodiment, the pour spout is formed along a corner of the bag, with a notch in the side of the bag located near the pour spout. The notch facilitates tearing of the bag to open the spout for pouring.

The inventive breastmilk bag is simple in fabrication, sanitary and disposable. It is flat, thus minimizing packaging, storage and transportation costs.

The breastmilk bag of this invention can be hermetically sealed, and thus remain sterile prior to use. In addition, after breastmilk is expressed into the bag, the bag opening can be re-sealed and the breastmilk can be sanitarily stored for later use. As previously noted, due to the provision of a writing area that is formed integral with the bag but is isolated from the liquid containing portion of the bag, there is no risk of puncturing the bag during writing, and writing is further facilitated by having a writing area that does not have liquid beneath it. When it is time to use the breastmilk, the breastmilk can easily and conveniently be poured from the bag by cutting or tearing the bag open at the pour spout.

The features and advantages of the present invention will be further understood upon consideration of the following detailed description of embodiments of the invention taken in conjunction with the accompanying drawings, in which:

### BRIEF DESCRIPTION OF THE DRAWINGS

FIG. 1 is a perspective view of a breastmilk bag made in accordance with the teachings of the present invention attached to a breastpump;

FIG. 2 is an exploded view of the breastmilk bag and breastpump of FIG. 1;

FIG. 3 is an enlarged plan view of the breastmilk bag of FIGS. 1 and 2 (with indicia removed);

FIG. 4 is a perspective view of a breastmilk bag substantially as shown in FIG. 3 re-sealed with a twisted tie; and

FIG. 5 is a plan view of another embodiment of a breastmilk bag made in accordance with the teachings of the present invention having a writing area that extends across the entire bottom of the bag.

### DETAILED DESCRIPTION OF THE EMBODIMENTS OF THE INVENTION

A breastpump useful in conjunction with the present invention is shown in U.S. Pat. No. 4,929,229. The disclosure of that patent is incorporated herein by reference. As will be readily recognized, however, the breastmilk bag of the present invention may be used with or adapted for many kinds of breastpumps.

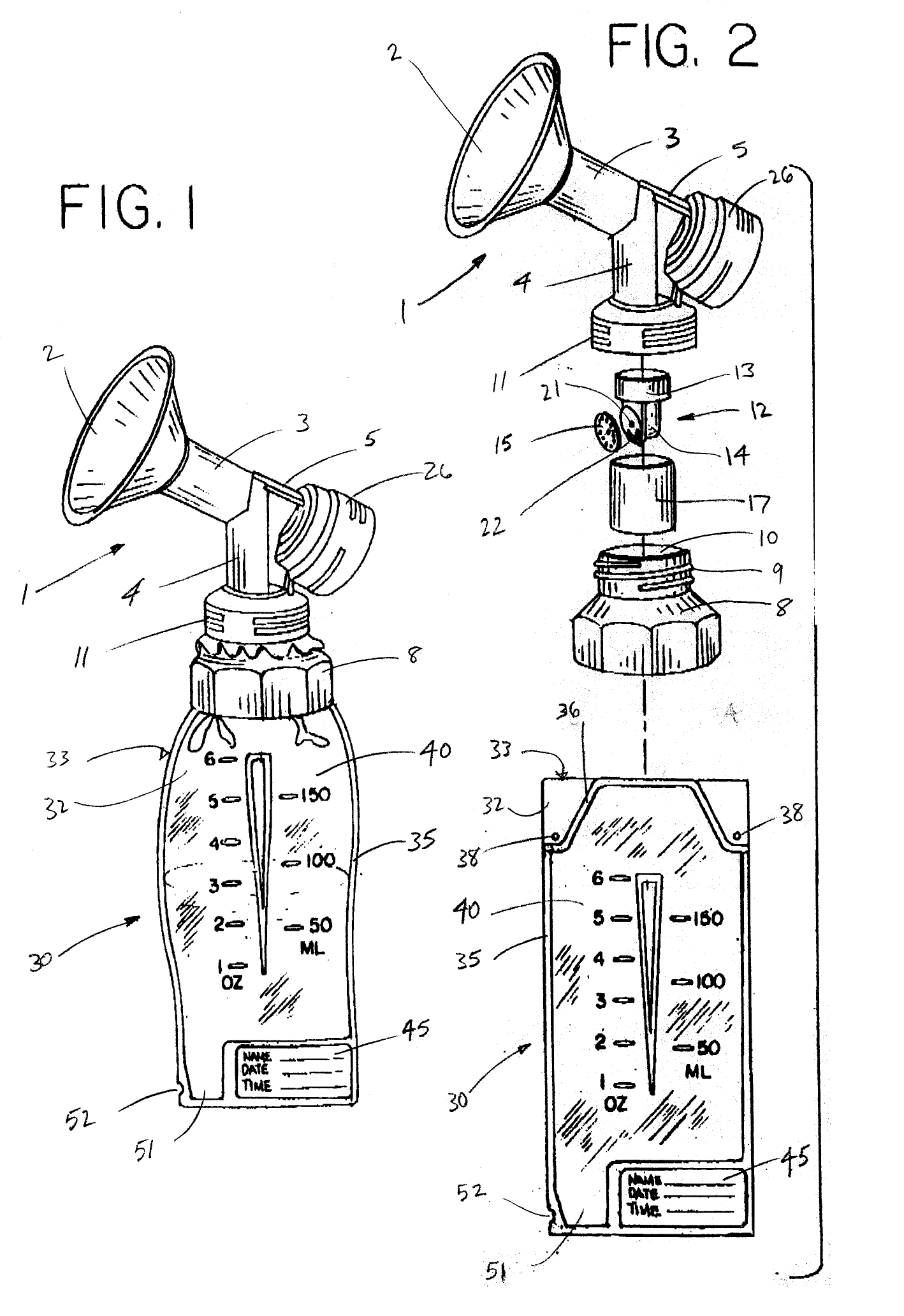

As seen in the accompanying FIGS. 1 and 2, the breastpump comprises a hood body or hood member 1 having two ends. The first end 2 is funnel shaped, and during use is placed over the breast of the user. A second end 3 of the hood member communicates with a collecting or catch chamber 4, and with a vacuum line (not shown) via an extension 5. Vacuum (or lower than ambient air pressure) can be provided by an electric or manual air pump (not shown), as described in U.S. Pat. No. 4,857,051. The breastpump has a threaded collar 11 which can be used with a compatibly threaded milk bottle neck, or as described hereafter with a milk bag adapter collar 8.

At the lower portion of the collecting chamber 4 is a valve mechanism. The valve generally consists of a rigid plastic housing 12 and a thin flexible membrane 15 made of rubber or silicone rubber. The valve housing 12 has an upper section 13 and a lower section 14. The upper section 13 is cylindrical in shape, and removably engages the outer portion of the outlet (not shown) to the catch chamber 4 of the breastpump in a friction fit. The thin flexible membrane 15 has a circular shape, and is attached to the lower portion 14 of the valve housing 12 by way of a knob (not shown) which is engaged in the opening 21 in a snap fit. The radius of the flexible membrane is large enough to completely cover the opening 22. Again, greater detail about the valve mechanism and its operation can be gleaned from U.S. Pat. No. 4,929,229.

A tubular sleeve 17 fits concentrically around valve housing 12 and is held in place via frictional engagement with the exterior of valve upper section 13. As will be seen hereafter, sleeve 17 prevents the breastmilk bag from interfering with the operation of the valve mechanism.

A bag 30 for containing breastmilk comprises a front sheet 32 and a back sheet 33. The front and back sheets 32, 33 are made of a suitable liquid impervious food compatible plastic, such as polyethylene. A polyethylenepolyester laminate can be advantageously used, with the polyethylene layer on the inside of the bag for flexibility, and also better sealability. Thermoplastic materials for making such disposable milk bags are well known, however. The sheets are joined by a seal 35, such as a heat seal, and a releasable seal 36. The heat seal 35 defines a writing area 45 and a liquid containing portion 40 for the bag 30. The bag 30 can similarly be formed from a continuous tube of plastic, eliminating the need for lateral seals for the bag.

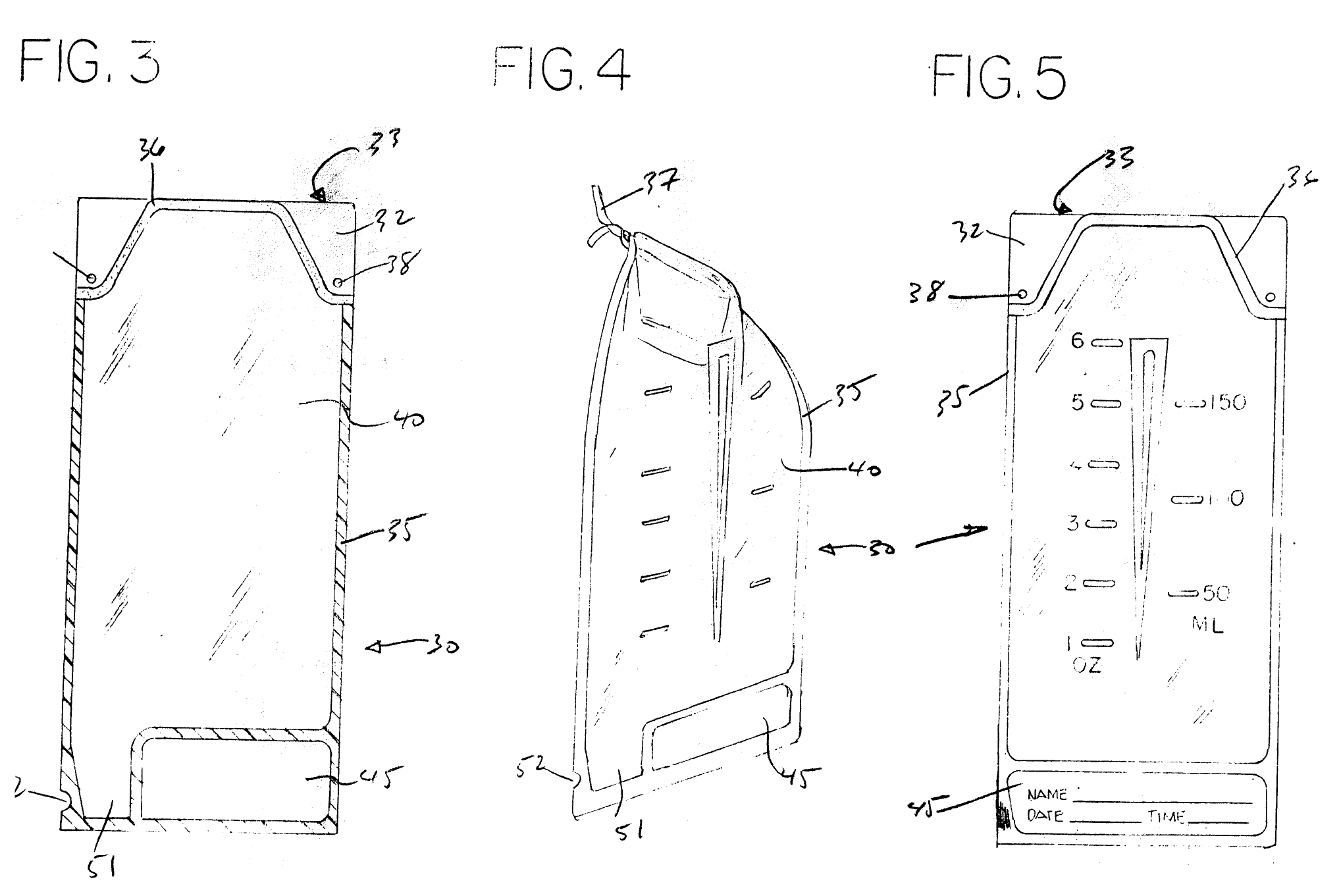

Writing area 45 is formed integral with the bag 30, but is isolated from the liquid containing portion 40. The writing area remains flat even when the bag 30 is filled. Advantageously, indicia can be provided on the writing area designating “Name”, “Date”, “Time” and the like. As shown in FIG. 5, one embodiment of the present invention has a writing area 45 that extends across the entire bottom of the breastmilk bag 30.

The releasable seal 36 is released or peeled-open by pulling the front and back sheets 32, 33 away from each other in the region of the seal 36. A suitable releasable seal can be formed by the so-called zone coating technique, whereby a material which will bond the two sheets 32, 33 together yet which is peelable is coated on one or both sheets in the area of what will be the seal 36. The seal 36 is then formed by setting the coating along the seal line. A suitable zone coating material for use with polyethylene is made by DRG Medical Packaging of Madison Wis., and is an ethyl vinyl acetate resin in a solvent base applied with a gravure cylinder in a technique well known in the art. Alternatively, a weak heat seal could be used to tack the sheets together in this region, or a weak adhesive seal could be used. Release of seal 36 forms an opening in the top of the bag for attachment of the bag to breastpump 1. Portions of sheets 32, 33 are not sealed in the corners of the bag adjacent the seal 36 to facilitate opening the bag. It will be noted that the material of the bag as well as the manner of effecting the seals are entirely matters of choice, and neither form a novel part of the invention claimed herein.

To attach bag 30 to breastpump 1, the open bag top is inserted through opening 10 in adapter collar 8, and the material of the front and back sheets 32, 33 at the opening is folded over the threaded portion 9 of adapter collar 8 in an apron-like manner. Breastpump 1 is then secured to adapter collar 8 via threaded collar 11, which engages compatible threads on the adapter collar 8 pinning the apron of the bag opening therebetween.

Breastmilk bag 30 is further provided with a tie 37, which fits in two holes 38 formed at substantially opposite sides of the releasable seal 36 that forms the bag opening. When the filled bag 30 is removed from breastpump 1, the bag is slid from the adapter collar 8 and re-sealed, as by folding down the top of the bag upon itself and then twisting or cinching the ends of tie 37.

As shown in FIGS. 1-4, breastmilk bag 30 is additionally provided with a pour spout 51. Pour spout 51 is formed integral with bag 30, being defined in this embodiment by a portion of the seal 35 of the liquid containing portion 40 which forms a side of the writing area 45. Pour spout 51 can be cut or clipped open to pour milk from the bag 30. In the preferred embodiment, however, bag 30 is additionally provided with a notch 52 at the periphery of the bag and adjacent to the pour spout 51. Pour spout 51 can thus be readily opened by tearing the bag at notch 52.

In use, milk is expressed from the breast of the user into the hood member 1, and then passes successively into catch chamber 4, valve housing 12, through hole 22, and into the breastmilk bag 30. When the user is finished, bag 30 is then removed from adapter collar 8, and the bag 30 can be re-sealed by passing the tie 37 through the holes 38, rolling down the bag in the area of the tie, and then twisting the tie together. Markings can be readily made on the writing area even after filling bag 30. The filled bag may then be stored for later use. At such later time, the milk is poured from the bag by either tearing the pour spout 51 open at notch 52, or by releasing tie 37. If a notch 52 is not provided, such as in FIG. 5, the bag 30 can simply be cut open.

Thus, while the invention has been described with reference to certain embodiments, those skilled in this art will recognize modifications of structure, arrangement, composition and the like that can be made to the present invention, yet will still fall within the scope of the invention as hereafter claimed.

What is claimed is:

1. A milk collecting device having a disposable container for use with a breastpump, comprising:
a hermetically sealable bag formed of milk impervious material for containing milk therein, said bag having an opening defined therein for use in attaching said bag to the breastpump, said opening being sealed by a releasable closure,
an adapter collar member having a opening defined therein through which adapter collar member opening said bag is received with material of said bag surrounding said bag opening being spread apart to overly said adapter collar member adjacent said adapter collar member opening,
the breastpump further including a breastpump collar for attachment of said adapter collar member thereto with said material of said bag being fixed between said breastpump collar and said adapter collar member for passage of milk through the breastpump and into said bag through said bag opening.

2. The milk collecting device of claim 1 further including a valve device for controlling fluid flow within the breastpump which valve device extends into said bag when said bag is attached to the breastpump collar, and a tubular sleeve surrounding said valve device to prevent interference with said valve by said bag material.

3. The milk collecting device of claim 1 wherein said adapter collar member and the breastpump collar are compatibly screw threaded for mutual attachment.

4. A bag for attachment to a breastmilk pump for containing breastmilk, comprising:
a liquid containing portion and a writing area, said writing area being formed integral with said bag and further being isolated from said liquid containing portion of said bag.

5. A bag for attachment to a breastmilk pump for containing breastmilk, comprising:
a milk container formed of a thin pliant liquid-impervious material, an opening provided in said milk container for attachment of said milk container to the breastmilk pump, and a spout separate from said opening formed integral with said milk container through which contained milk can be poured from the milk container.

6. A bag attachable to a breastmilk pump for containing breastmilk, said bag being hermetically sealed having an opening defined therein, which opening is closed by releasable means to seal said opening such that said opening can be opened for attachment of said bag to the breastmilk pump.

7. A bag for attachment to a breastmilk pump for containing breastmilk, comprising:
a milk container made of thin pliant liquid-impervious material,
a tie,
said milk container having at least two holes for receiving said tie substantially at opposite sides of an opening defined in said milk container for attachment of said milk container to said breastmilk pump, said tie when fit through said holes and cinched thereby sealing said opening when said bag is removed from the breastmilk pump.

8. A bag for attachment to a breastmilk pump for containing breastmilk comprising:
a thermoplastic film front sheet and a thermoplastic film back sheet, said front and back sheets being joined by a first seal, said first seal being a permanent seal and defining a liquid containing portion,
a writing area formed integral with said bag by said first seal and further being isolated from said liquid containing portion, and
a pour spout formed integral with said bag and defined by said first seal, said pour spout being separate from an opening defined by said front and back sheets which opening is used for attachment of said bag to the breastpump.

9. The bag of claim 8 further including a notch formed at the periphery of said bag and adjacent to said pour spout to tear said bag at said pour spout so that contained milk can be poured from the bag.

10. The bag of claim 8 further having a second seal, said second seal being releasable and defining an opening in said liquid containing portion for attachment of said bag to the breastmilk pump, said second seal being peeled open to form said opening.

11. The bag of claim 10 further having a tie and at least two holes formed in said bag for receiving said tie at substantially opposite sides of said opening, whereby said tie when fit through said holes and cinched closes said opening when said bag is removed from the breastmilk pump.

12. A bag for attachment to a breastmilk pump for containing breastmilk comprising:
a plastic film front sheet and a plastic film back sheet, said front and back sheets being joined by a first seal line, said first seal line being a permanent seal and defining a liquid containing portion,
a second seal line joining said front and back sheets, said second seal line being releasable and defining an opening in said liquid containing portion for attachment of said bag to the breastmilk pump, said second seal line being peeled apart to form said opening, and
a writing area formed integral with said bag by said first seal line and further being isolated from said liquid containing portion.

13. The bag of claim 12 having a pour spout formed integral with said bag and being defined by said first seal, a notch being formed at the periphery of said bag and adjacent to said pour spout to tear said bag at said pour spout so that contained milk can be poured from said bag, said writing area having a common side with said pour spout formed by said first seal line.

14. The bag of claim 12 further including a tie, and at least two holes formed in said bag for receiving said tie at substantially opposite sides of said opening, whereby said tie when fit through said holes and cinched closes said opening when said bag is removed from the breastmilk pump.

15. A bag for attachment to a breastmilk pump for containing breastmilk, comprising:
a thermoplastic front sheet and a thermoplastic back sheet, said front and back sheets being joined by a first permanent seal and defining a liquid containing portion,
a second releasable seal defining an opening in said liquid containing portion for attachment of said bag to the breastmilk pump, said second seal being peeled open to form said opening,
a pour spout formed integral with said bag and being defined by said first seal, a notch being formed at the periphery of said bag and adjacent to said pour spout to tear said bag at said pour spout so that contained milk can be poured from said bag.

16. A milk collecting device having a disposable container for use with a breastpump, comprising:
a thermoplastic bag for containing milk therein, said bag having a releasable seal defining an opening for use in attaching said bag to the breastpump, and a permanent seal defining a writing area formed integral with said bag which writing area is isolated from milk contained within said bag, said permanent seal further defining a pour spout formed integral with said bag but separate from said bag opening,
an adapter collar member having an opening defined therein through which adapter collar member opening said bag is received with part of said bag surrounding said bag opening being spread apart in overlying fashion with respect to said adapter collar opening,
the breastpump further including a breastpump collar for attachment of said adapter collar member thereto with said part of said bag surrounding said bag opening being fixed between said breastpump collar and said adapter collar member for passage of milk through the breastpump and into said bag through said bag opening, and
a tie for use in closing said bag opening, said bag material having a pair of holes defined therein adjacent said bag opening and on opposite sides of said bag opening through which holes said tie is received.
